(19) United States
(12) Patent Application Publication
(10) Pub. No.: US 2018/0177994 A1
(43) Pub. Date: Jun. 28, 2018

(54) CONNECTION DEVICE FOR A CATHETER, IN PARTICULAR A PERIPHERAL VENOUS CATHETER (III)

(71) Applicant: SICOS GMBH, Duesseldorf (DE)

(72) Inventors: André Reichardt, Apolda (DE); Christian Frauenrath, Juelich (DE); Kai Markus, Eschweiler (DE)

(21) Appl. No.: 15/309,497

(22) PCT Filed: Apr. 30, 2015
(86) PCT No.: PCT/DE2015/100179

§ 371 Date: Sep. 20, 2017

(30) Foreign Application Priority Data
May 8, 2014 (DE) 10 2014 106 458.5

(51) Int. Cl. A61M 39/16

(52) U.S. Cl. CPC A61M 39/162

(57) ABSTRACT

A connection device for a catheter, especially for a peripheral venous catheter, comprising
a) a main body (1) having a main body infusion channel (2), the main body with a through-flow element (3, 103, 203), having sterilizing effect and forming at least a part of the main body infusion channel (2, 102), and with a coupling element (6), wherein the main body (1) is in one piece, and
b) an attachment body (8) having an attachment body infusion channel (7), wherein the attachment body (8) and the coupling element (6) have means to detachably fix the attachment body (8) to the coupling element (6),
is characterized in that
c) the main body (1) comprises massive material having a sterilizing effect, wherein the massive material is uncoated at least on walls provided for contacting with infusion liquid and
d) the massive material comprises a matrix made of at least one first material, wherein at least one further material having a sterilizing effect is incorporated into the matrix.

The invention relates to a connection device for a catheter, especially for a peripheral venous catheter, comprising a main body, having a main body infusion channel, the main body with a through-flow element, having sterilizing effect and forming at least a part of the main body infusion channel, and with a coupling element, wherein the main body is in one piece. In addition, the connection device comprises an attachment body having an attachment body infusion channel, wherein the attachment body and the coupling element have means to detachably fix the attachment body to the coupling element.

A connection device is known from EP 1 579 878 B1, which is particularly suitable for central venous catheters, which remain on the human body for long periods of time, in order to reduce the risk of septa caused by the entry of bacteria. Central venous catheters can be used e.g. for the artificial diet at short-bow syndrome. Such septa are associated with considerable personal disadvantages for the treated person. As a solution, the above-mentioned prior art proposes to provide a sterilizing through-flow element in form of a sieve element being made of silver, having numerous openings and being arranged in the infusion channel of the connection device disclosed there or, seen in the direction of the infusion flow, at the end of the infusion channel. The sieve element represents a barrier for bacteria and/or fungi and is also advantageous for the reason that the complete connection device together with the sieve element can be replaced in a very simple manner, in case the sieve element should become ineffective, e.g. as a result of clogging.

U.S. Pat. No. 5,782,808 discloses a connection device of the type initially mentioned in which the main body, comprising the through-flow element, is integral. It is also disclosed to provide various surfaces of the main body as well as surfaces of the attachment body, which can be connected to the main body via a thread, with a sterilizing coating. In one embodiment, it is furthermore possible to provide also the inner surfaces of the through-flow element with the silver coating. For the sealing between main body and attachment body sealing rings are provided. It is laborious to reliably affix a complete silver coating, in particular on the inner surfaces of the through-flow element.

U.S. Pat. No. 5,049,139 discloses a connection device for a catheter in which a block of a water-soluble glass that is doped with silver, is arranged between two parts of the connection device, and has a passage for the infusion liquid. During deployment parts of the block dissolve in the infusion liquid so that the antibacterial properties of the silver can come into effect.

It is an object of the present invention to provide a connection device of the type mentioned at the outset which is easier to produce and which can further improve the protective effect against sepses.

This object is achieved in a connection device of the type mentioned at the outset by the feature that the main body comprises massive material having a sterilizing effect, wherein the massive material is uncoated at least on walls provided for contacting with infusion liquid and wherein the massive material comprises a matrix made of at least one first material, wherein at least one further material having a sterilizing effect is incorporated into the matrix.

Exemplary and advantageous embodiments of the connection device are defined in the dependent claims.

A massive material is understood to be a material which has a shape stability without being applied to a carrier or a supporting substrate. Foils or thin films applied to a substrate are thus not massive material. However, a massive material can be a matrix doped with foreign substances or comprising foreign elements, for example reinforcing elements, in particular fibers.

Manufacturing the integral main body from a sterilizing massive material can make a coating, in particular a coating with a sterilizing effect, redundant. The massive material comprises a matrix of at least one first material, wherein into the matrix at least one further material having a sterilizing effect is incorporated, for example in the form of an additive or doping. The at least one matrix material, which can itself be without or with a sterilizing effect, can be, for example, a plastic. It is particularly advantageous if the entire main body consists of a uniform plastic material as a matrix with a sterilizing doping.

Especially, those agents and substances are to be understood as sterilizing which are in particular bactericidal, antibiotic and/or cytostatic. Applicable as a sterilizing agent or as a sterilizing substance are e.g. silver, copper or silver- and/or copper-containing alloys or silver- and/or copper-containing chemical compounds.

It can be advantageous to configure the connection device according to the invention such that the through-flow element has a liquid-tight circumferential wall and in an end wall at least one outlet opening, wherein the through-flow element undergoes a sealing press fit with the attachment body infusion channel when the attachment body is fixed to the main body. On these grounds an infusion liquid can enter the attachment infusion channel only through the at least one outlet opening of the end wall. With the sealing press fit between the through-flow element and the attachment body infusion channel, the tightness of the connection between the main body and the attachment body with respect to the infusion liquid can be ensured. By this means further sealing measures can be supported or become dispensable. In particular, a sealing press fit between the main body and the attachment body can be provided as a further sealing measure.

With the sealing press fit between through-flow element and attachment body infusion channel, production tolerances on the dimensions of the attachment body infusion channel can be absorbed to some extent.

In order to achieve the press fit, the through-flow element and the inner wall of the attachment body infusion channel have suitable shapes. For example, the inner wall of the attachment body infusion channel can taper in the direction of flow of the infusion liquid, for example, the inner wall can be formed conically with a certain opening angle at least in the area of the intended contact with the sterilizing through-flow element. In this case, the through-flow element, at least in the region of the expected contact with the inner wall of the attachment body infusion channel, may have a cylindrically shaped outer wall or as well taper in the direction of flow, wherein the given per unit of length diameter reduction of the outer side of the circumferential wall of the through-flow element is less than the given per unit length diameter reduction of the inner wall of the attachment body infusion channel. In the case of a conical shape, the outer side of the circumferential wall of the through-flow element has a smaller opening angle than the inner wall of the attachment body infusion channel.

To provide an elastic material for the through-flow element and/or for the attachment body infusion channel can be advantageous for the press fit.

The main body and the attachment body can be fixed to each other e.g. via threads.

The through-flow element can have a single outlet opening or two or more outlet openings in its end wall. A plurality of small outlet openings can also be provided, whereby the through-flow element has a sieve-like construction. Provided a liquid tightness of the circumferential wall of the through-flow element is not required, also the circumferential wall can have at least one outlet opening.

The connection device according to the invention can also be configured in such a way that at least one sterilizing insert body is arranged in the interior of the through-flow element. The at least one insert body increases the sterilizing surface and can be used independently of the number of outlet openings. The at least one insert body can e.g. have a cruciform cross-section viewed in the direction of flow. The at least one insert body in the interior of the through-flow element can be separate and interchangeable or likewise integral with the through-flow element and the main body. The at least one insert body can be made of the same material as the main body. However, the insert body or at least one of the insert bodies can also consist of a material other than that of the main body. As material of the at least one insert body, for example massive silver or copper, or a massive alloy containing silver and/or copper, or a sterilizing doped plastic may be suitable, wherein in case of several insert bodies these may consist of different materials.

The attachment body infusion channel may also have a sterilizing effect, for example by means of a suitable coating or sterilizing agents or substances which have been incorporated, for example by doping, into the wall delimiting the attachment body infusion channel. As a result, the sterilizing surface that is in direct contact with the liquid flowing through is enlarged, which can mean increased protection. In particular, the attachment body can comprise a sterilizing massive material, wherein all material variants of the massive material described in connection with the main body can also be advantageous for the attachment body. In addition, the attachment body can be designed in one piece.

The connection device according to the invention can also be configured in such a way that the attachment body has, at its end facing away from the main body, a coupling piece for connection to a cannula, in particular a butterfly cannula. In this way, the application of a catheter can be speeded up and simplified. Intermediate elements, for example, in the form of a hose, which may also be contaminated and the attachment of which is time consuming, become dispensable therewith.

However, it may also be advantageous to design the connection device according to the invention in such a way that the attachment body is part of a cannula, in particular a butterfly cannula.

In the following preferred embodiments of the connection device according to the invention are presented on the basis of figures.

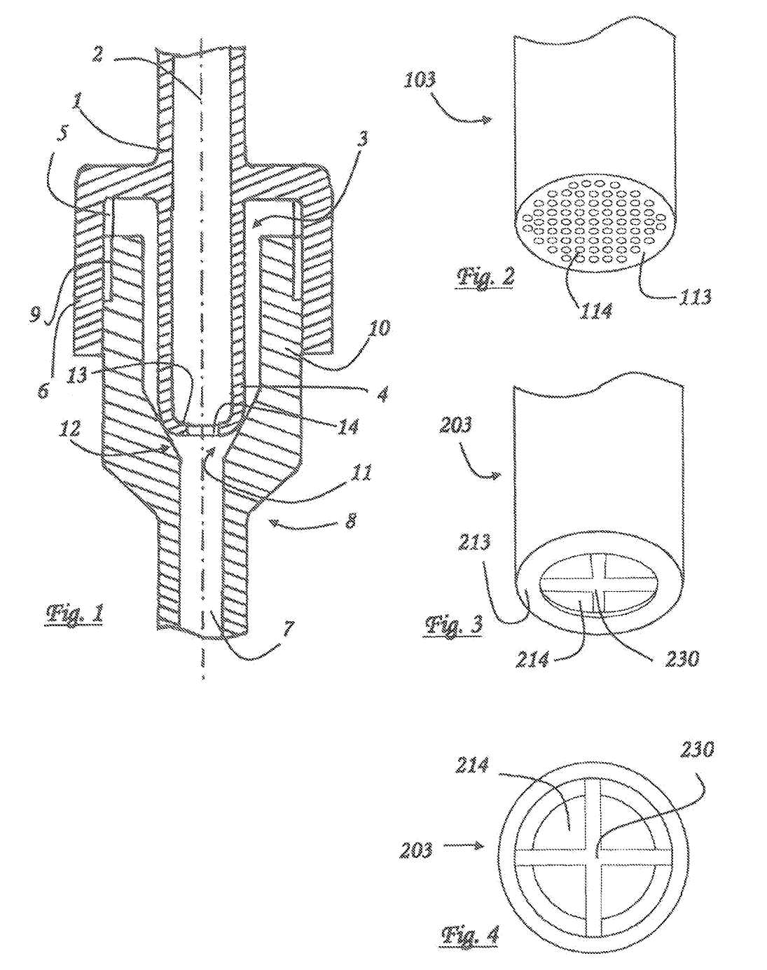

FIG. 1 shows schematically a connection device with main body and attachment body,

FIG. 2 shows schematically an end piece of a first through-flow element in a perspective view,

FIG. 3 shows schematically an end piece of a second through-flow element in a perspective view, and

FIG. 4 shows schematically the second through-flow element in a top view.

FIG. 1 shows in schematic representation a connection device with a main body 1 through which a main body infusion channel 2 passes. In a manner not shown here the main body 1 is connected to an element that delivers infusion liquid e.g. an infusion hose, which is also not shown here. The main body 1 has a through-flow element 3 that delimits a part of the infusion channel 2 and the circumferential wall 4 of which is designed liquid-tight. The main body 1 is formed in one piece and consists of a massive material with a plastic matrix having a sterilizing doping, e.g. comprising silver.

An attachment body 8, having an attachment body infusion channel 7, is fixed to the main body 1 via a coupling sleeve 6 of the main body 1, the coupling sleeve having a thread 5 and surrounding a part of the through-flow element 3. For this purpose, an engagement sleeve 10, belonging to the attachment body 8 and having a thread 9, is screwed into the coupling sleeve 6. When screwing the attachment body 10 in, an end piece 11 of the through-flow element 3 enters into a liquid-tight press fit with a conical inlet part 12 of the attachment body 8. The lower end wall 13 of the through-flow element 3 has an outlet opening 14 for the flow of the infusion liquid.

The attachment body 8 can be connected to a discharging infusion tube (not shown here) or to a cannula (not shown here). The attachment body 8 can also be part of the cannula.

FIG. 2 shows the end piece of a through-flow element 103 with an alternative shape in comparison to through-flow element 3 of FIG. 1. Through-flow element 103 has an end wall 13 with a plurality of outlet openings so that a sieve-like structure is formed.

As a further embodiment FIG. 3 shows a perspective view of the end region of a through-flow element 203 with one outlet opening 214 in an end wall 213. Within the flow element, an insert body 230 is arranged, which also has a sterilizing effect and thus increases the surface acting on the infusion fluid. The insert body 230 has a cruciform cross-section in the flow direction and divides the outlet opening 214 into four partial openings. FIG. 4 shows the flow element 203 with insert body 230 in a top view subsequent to a cut along a plane perpendicular to the longitudinal direction.

Other forms of one or more insert bodies are also possible.

The insert body can be detachably inserted as a separate element. However, the insert body can also be integral with the through-flow element 203 and thus with the remaining main body 1 (FIG. 1).

In FIGS. 2 and 3, the transition from the respective circumferential wall to the end wall 113 or 213, respectively, is reproduced with a 90°-edge for simplifying the illustration. This is one possible variant. However, the transition to the end wall 113 or 213, respectively, can have any shape which allows a press fit with the attachment piece 8, for example, similar to or rounded as in FIG. 1, or further alternatively also cone-shaped.

### LIST OF REFERENCE NUMBERS

1 Main body

2 Main body infusion channel

3 Through-flow element

4 Circumferential wall

5 Thread

6 Coupling sleeve

7 Attachment body infusion channel

8 Attachment body

9 Thread

10 Engagement sleeve

11 End piece

12 Inlet part

13 End wall

14 Outlet opening

103 Through-flow element

113 End wall

114 Outlet opening

203 Through-flow element

213 End wall

214 Outlet opening

230 Insert body

1-10. (canceled)

11. A connection device for a catheter, especially for a peripheral venous catheter, comprising
a main body, having a main body infusion channel, the main body with a through-flow element, having sterilizing effect and forming at least a part of the main body infusion channel, and with a coupling element, wherein the main body is in one piece, and
an attachment body having an attachment body infusion channel, wherein the attachment body and the coupling element have means to detachably fix the attachment body to the coupling element,
wherein,
the main body comprises massive material having a sterilizing effect, wherein the massive material is uncoated at least on walls provided for contacting with infusion liquid and
the massive material comprises a matrix made of at least one first material, wherein at least one further material having a sterilizing effect is incorporated into the matrix.

12. The connection device claimed in claim 11 wherein the at least one first material of the matrix is a plastic or a plastic mixture.

13. The connection device claimed in claim 11 wherein the through-flow element has a liquid-tight circumferential wall and in an end wall at least one outlet opening, wherein
the through-flow element undergoes a sealing press fit with the attachment body infusion channel when the attachment body is fixed to the main body.

14. The connection device claimed in claim 11 wherein the attachment body infusion channel tapers in the direction of flow at least in an inlet part surrounding the through-flow element.

15. The connection device claimed in claim 14 wherein the outer side of the circumferential wall of the through-flow element is cylindrical.

16. The connection device claimed in claim 14 wherein the outer side of the circumferential wall of the through-flow element is conical with a diameter decreasing towards the end wall.

17. The connection device claimed in claim 11 wherein at least one sterilizing insert body is arranged inside the through-flow element.

18. The connection device claimed in claim 14 wherein at least one sterilizing insert body is arranged inside the through-flow element.

19. The connection device claimed in claim 15 wherein at least one sterilizing insert body is arranged inside the through-flow element.

20. The connection device claimed in claim 16 wherein at least one sterilizing insert body is arranged inside the through-flow element.

21. The connection device claimed in claim 11 wherein the attachment body has a sterilizing effect, at least on a section of the attachment body infusion channel.

22. The connection device claimed in claim 11 wherein the attachment body has, at its end facing away from the main body, a coupling piece for connection to a cannula, in particular a butterfly cannula.

23. The connection device claimed in claim 11 wherein the attachment body is part of a cannula, in particular of a butterfly cannula.
